(19) United States
(12) Patent Application Publication
(10) Pub. No.: US 2026/0026856 A1
(43) Pub. Date: Jan. 29, 2026

(54) MEDICAL DEVICE WITH AXIAL STABILITY FOR LACERATING CARDIAC VALVE LEAFLETS

(71) Applicant: BOSTON SCIENTIFIC SCIMED, INC., Maple Grove, MN (US)

(72) Inventors: Eric Michael Petersen, Maple Grove, MN (US); Joel T. Eggert, Plymouth, MN (US); Joseph Edward Adriaens, Elk River, MN (US); Daniel Presteng, Corcoran, MN (US); James M. Anderson, Corcoran, MN (US); Philip Andrew Litecky, Forest Lake, MN (US)

(73) Assignee: BOSTON SCIENTIFIC SCIMED, INC., MAPLE GROVE, MN (US)

(21) Appl. No.: 19/272,944

(22) Filed: Jul. 17, 2025

Related U.S. Application Data
(60) Provisional application No. 63/674,909, filed on Jul. 24, 2024.

(51) Int. Cl. A61B 18/08; A61B 18/00

(52) U.S. Cl. CPC A61B 18/082; A61B 2018/00077; A61B 2018/00184; A61B 2018/0022; A61B 2018/00369; A61B 2018/00595; A61B 2018/00601

(57) ABSTRACT

A medical device may be used for lacerating valve leaflets and includes an elongate shaft extending proximally from a distal region, an expandable element that is secured within the distal region and is movable between a collapsed configuration and an expanded configuration, and a conductive member including an electrocautery electrode portion extending over the expandable element. The electrocautery electrode portion has a first length when the expandable element is in the collapsed configuration and a second length greater than the first length when the expandable element is in its expanded configuration. The conductive member is adapted to return the electrocautery electrode portion to the first length when the expandable element returns to the collapsed configuration.

### CROSS REFERENCE TO RELATED APPLICATIONS

This application claims the benefit of priority of U.S. Provisional Application No. 63/674,909 filed Jul. 24, 2024, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, for example, intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for lacerating valve leaflets. The medical device includes an elongate shaft that extends proximally from a distal region and an expandable element that is secured within the distal region. The expandable element is movable between a collapsed configuration and an expanded configuration. A conductive member includes an electrocautery electrode portion extending over the expandable element. The electrocautery electrode portion has a first length when the expandable element is in the collapsed configuration and has a second length greater than the first length when the expandable element is in its expanded configuration. The conductive member is adapted to return the electrocautery electrode portion to the first length when the expandable element returns to the collapsed configuration.

Alternatively or additionally, the conductive member may include an undulating region that is adapted to elongate in order to accommodate a change from the first length of the electrocautery electrode portion to the second length of the electrocautery electrode portion when the expandable element expands into the expanded configuration.

Alternatively or additionally, the undulating region may be adapted to contract in order to accommodate a change from the second length of the electrocautery electrode portion to the first length of the electrocautery electrode when the expandable element returns to the collapsed configuration.

Alternatively or additionally, a distal region of the electrocautery electrode portion may be secured to a distal region of the expandable element.

Alternatively or additionally, the undulating region may be disposed proximal of the expandable element.

Alternatively or additionally, a distal region of the electrocautery electrode portion may be secured to a distal region of the expandable element and a proximal region of the electrocautery electrode portion may be secured to a proximal region of the expandable element.

Alternatively or additionally, the conductive member may include a first undulating region that is adjacent to the distal region of the electrocautery electrode portion and a second undulating region that is adjacent to the proximal region of the electrocautery electrode portion.

Alternatively or additionally, the medical device may further include a sled that is slidingly disposed relative to the elongate shaft and a spring that biases the sled to a proximal position. The conductive member may be operably coupled with the sled such that the sled may move distally against a biasing force provided by the spring when the expandable element expands into the expanded configuration.

Alternatively or additionally, the sled may move proximally when the expandable element returns to the collapsed configuration.

Alternatively or additionally, the medical device may further include a proximal hub that is secured to a proximal region of the elongate shaft and a spool that is disposed relative to the proximal hub. The spool is adapted to releasably wind up a portion of the conductive member on the spool.

Alternatively or additionally, the spool may be disposed inside the proximal hub.

Alternatively or additionally, the spool may be disposed outside of the proximal hub.

Alternatively or additionally, the expandable element may include an inflatable balloon.

Another example may be found in a medical device for lacerating valve leaflets. The medical device includes an elongate shaft that extends proximally from a distal region and an inflatable balloon that is secured within the distal region and is adapted to be inflated and/or deflated. An electrocautery electrode extends over the inflatable balloon. A conductive member extends proximally from the electrocautery electrode. A biasing member is adapted to accommodate a change in length of the electrocautery electrode when the inflatable balloon is either inflated or deflated.

Alternatively or additionally, the biasing member may include an undulating region of the conductive member.

Alternatively or additionally, the biasing member may further include a second undulating region of the conductive member.

Alternatively or additionally, the biasing member may include a sled that is slidingly disposed relative to the elongate shaft and a spring that biases the sled to a proximal position. The conductive member may be operably coupled with the sled such that the sled moves distally against a biasing force provided by the spring when the inflatable balloon is inflated.

Alternatively or additionally, the medical device may further include a proximal hub that is secured to a proximal region of the elongate shaft. The biasing member may include a spool that is disposed relative to the proximal hub and is adapted to releasably wind up a portion of the conductive member on the spool.

Another example may be found in a medical device for lacerating valve leaflets. The medical device includes an elongate shaft that extends proximally from a distal region and an inflatable balloon that is secured within the distal region. The inflatable balloon is adapted to be inflated and/or deflated. A conductive member extends relative to the elongate shaft and includes an electrocautery electrode portion that extends over the inflatable balloon and an undulating region that is adapted to elongate in order to accommodate an increase in length of the electrocautery electrode portion when the inflatable balloon is inflated.

Alternatively or additionally, the undulating region may be adapted to decrease in length in order to accommodate a decrease in length of the electrocautery electrode portion when the inflatable balloon is deflated.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing a replacement heart valve implant positioned within a native valve annulus of a heart;

FIG. 2A is a schematic view of an illustrative medical device shown with an expandable element in a collapsed configuration;

FIG. 2B is a schematic view of the illustrative medical device of FIG. 2A, shown with the expandable element in an expanded configuration;

FIG. 3A is a schematic view of an illustrative medical device shown with an expandable element in a collapsed configuration;

FIG. 3B is a schematic view of the illustrative medical device of FIG. 3A, shown with the expandable element in an expanded configuration;

FIG. 4A is a schematic view of an illustrative medical device shown with an expandable element in a collapsed configuration;

FIG. 4B is a schematic view of the illustrative medical device of FIG. 4A, shown with the expandable element in an expanded configuration;

FIG. 5 is a schematic view of an illustrative medical device having an internal spool;

FIG. 6 is a schematic view of an illustrative medical device having an external spool;

FIG. 7A is a schematic view of an illustrative medical device shown with an expandable element in a collapsed configuration; and

FIG. 7B is a schematic view of the illustrative medical device of FIG. 7A, shown with the expandable element in an expanded configuration.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### Description

The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to place a stent, or perform balloon angioplasty, in one of the coronary arteries. In some instances, it may be beneficial to slice or lacerate one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking cardiac artery ostiums when displayed to the side when a second artificial heart valve is implanted in place of the first artificial heart valve. The artificial valve leaflets forming part of the artificial heart valve, which may for example be made from porcine or bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

A medical device for lacerating valve leaflets includes an elongate shaft that extends proximally from a distal region and an expandable element that is secured within the distal region and is movable between a collapsed configuration and an expanded configuration. A conductive member includes an electrocautery electrode portion that extends over the expandable element. The electrocautery electrode portion has a first length when the expandable element is in the collapsed configuration and has a second length greater than the first length when the expandable element is in its expanded configuration. The conductive member is adapted to return the electrocautery electrode portion to the first length when the expandable element returns to the collapsed configuration. In some cases, the expandable element may be an inflatable balloon.

In some cases, the conductive member includes an undulating region that is adapted to elongate in order to accommodate a change from the first length of the electrocautery electrode portion to the second length of the electrocautery electrode portion when the expandable element expands into the expanded configuration. The undulating region may be adapted to contract in order to accommodate a change from the second length of the electrocautery electrode portion to the first length of the electrocautery electrode when the expandable element returns to the collapsed configuration. In some cases, a distal region of the electrocautery electrode portion may be secured to a distal region of the expandable element. In some cases, the undulating region may be disposed proximal of the expandable element.

In some cases, a distal region of the electrocautery electrode portion may be secured to a distal region of the expandable element and a proximal region of the electrocautery electrode portion may be secured to a proximal region of the expandable element. In some cases, the conductive member may include a first undulating region that is adjacent to the distal region of the electrocautery electrode portion and a second undulating region that is adjacent to the proximal region of the electrocautery electrode portion.

In some cases, the medical device may further include a sled that is slidingly disposed relative to the elongate shaft and a spring that biases the sled to a proximal position. The conductive member may be operably coupled with the sled such that the sled may move distally against a biasing force provided by the spring when the expandable element expands into the expanded configuration. In some cases, the sled may move proximally when the expandable element returns to the collapsed configuration.

In some cases, the medical device may further include a proximal hub that is secured to a proximal region of the elongate shaft and a spool that is disposed relative to the proximal hub. The spool may be adapted to releasably wind up a portion of the conductive member on the spool. In some cases, the spool may be disposed inside the proximal hub. In some cases, the spool may be disposed outside of the proximal hub.

A medical device for lacerating valve leaflets may include an elongate shaft that extends proximally from a distal region, with an inflatable balloon that is adapted to be inflated and/or deflated that is secured within the distal region. An electrocautery electrode extends over the inflatable balloon. A conductive member extends proximally from the electrocautery electrode. A biasing member is adapted to accommodate a change in length of the electrocautery electrode when the inflatable balloon is either inflated or deflated.

In some cases, the biasing member may include an undulating region of the conductive member. In some cases, the biasing member may further include a second undulating region of the conductive member. In some cases, the biasing member may include a sled that is slidingly disposed relative to the elongate shaft and a spring that biases the sled to a proximal position. The conductive member may be operably coupled with the sled such that the sled may move distally against a biasing force provided by the spring when the inflatable balloon is inflated. In some cases, the medical device may further include a proximal hub that is secured to a proximal region of the elongate shaft. The biasing member may include a spool that is disposed relative to the proximal hub and is adapted to releasably wind up a portion of the conductive member on the spool.

A medical device for lacerating valve leaflets may include an elongate shaft extending proximally from a distal region and an inflatable balloon that is secured within the distal region and is adapted to be inflated and/or deflated. A conductive member extends relative to the elongate shaft and includes an electrocautery electrode portion that extends over the inflatable balloon and an undulating region that is adapted to elongate in order to accommodate an increase in length of the electrocautery electrode portion when the inflatable balloon is inflated. In some cases, the undulating region may be adapted to decrease in length in order to accommodate a decrease in length of the electrocautery electrode portion when the inflatable balloon is deflated.

As noted, the medical devices described herein may be used in lacerating portions of valve leaflets regardless of whether the valve leaflets are native valve leaflets or artificial valve leaflets. In some cases, portions of the native valve leaflets may be lacerated prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to lacerate one or more of the artificial valve leaflets within the previously implanted artificial heart valve before implanting the replacement artificial heart valve within the previously implanted artificial heart valve. In some cases, lacerating one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

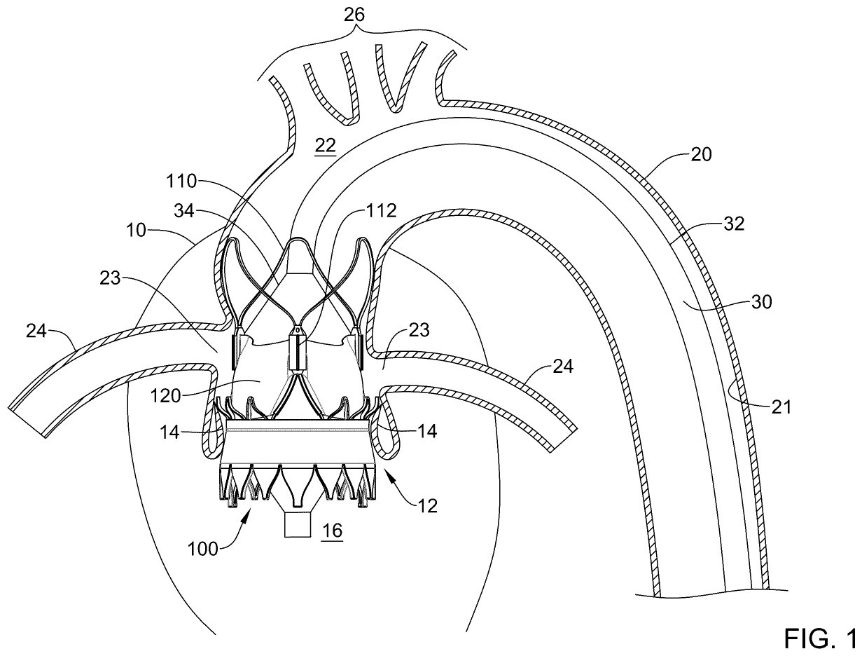

FIG. 1 is a schematic partial cut-away view of a portion of a patient's heart 10 including an aortic valve 12 having native valve leaflets 14 disposed within and/or extending from the native valve annulus, a left ventricle 16, and certain connected vasculature, such as an aorta 20 connected to the aortic valve 12 of the patient's heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

FIG. 1 further illustrates selected features of a replacement heart valve implant 100 positioned within the aortic valve 12 and/or the native valve annulus of the aortic valve 12. Some non-limiting examples of the replacement heart valve 100 may include the ACURATE NEO2™, the ACURATE PRIME™, and/or family members thereof from Boston Scientific. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some instances, the expandable framework 110 may be self-expanding. In some instances, the expandable framework 110 may be self-biased toward the radially expanded configuration. In some cases, the expandable framework 110 may be mechanically expandable. As an example, the expandable framework 110 may be balloon expandable.

In some instances, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some cases, the plurality of valve leaflets 120 may include a polymer such as a thermoplastic polymer. In some cases, the plurality of valve leaflets 120 may include at least 50 percent by weight of a polymer. In some instances, the plurality of valve leaflets 120 may be formed from porcine pericardium, bovine pericardium, or other living tissue. Other configurations and/or materials are also contemplated.

As seen in FIG. 1, a medical device 30 extends through the aortic arch 22 and into an interior of the previously implanted replacement heart valve implant 100. As will be described, the medical device 30 may be used for lacerating at least part of one or more of the valve leaflets 120 of the replacement heart valve implant 100. The medical device 30 includes an elongate shaft 32 that extends through the aortic arch 22. The medical device 30 includes an expandable element 34. In some cases, the expandable element 34 may be an expandable stent, for example. In some cases, as shown, the expandable element 34 is an inflatable balloon.

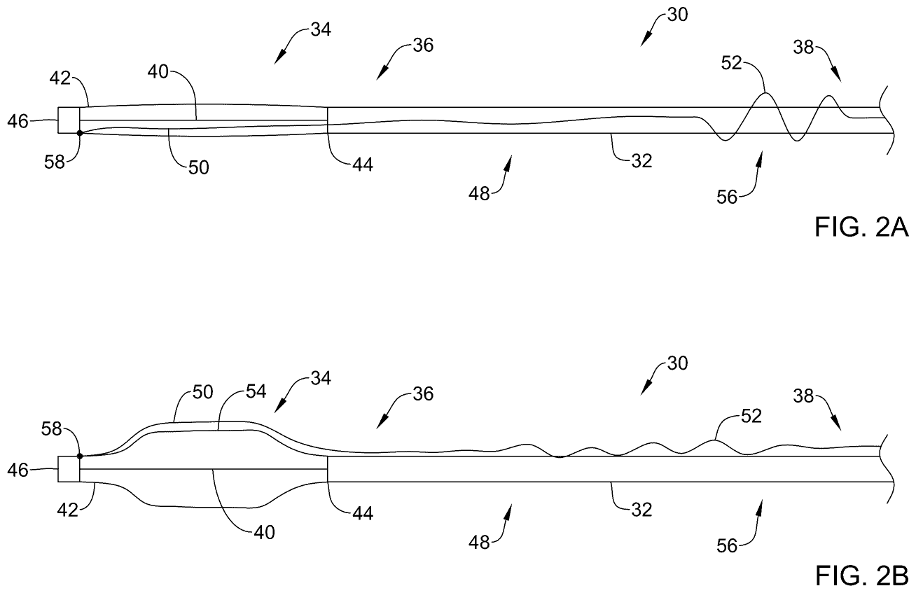

FIG. 2A and FIG. 2B are schematic views of the medical device 30. FIG. 2A shows the expandable element 34 in a collapsed configuration and FIG. 2B shows the expandable element 34 in an expanded configuration. The elongate shaft 32 includes a distal region 36 and extends proximally to a proximal region 38. The expandable element 34 may be secured to the distal region 36 of the elongate shaft 32. In some cases, a guidewire lumen 40 may be seen as extending distally of the elongate shaft 32 and extending through the expandable element 34. While not expressly shown, it will be appreciated that the guidewire lumen 40 may extend proximally through the elongate shaft 32. In some cases, additional lumens (not shown) may also extend through the elongate shaft 32. As an example, an inflation lumen may extend through the elongate shaft 32, particularly when the expandable element 34 is an inflatable balloon. In some cases, while not shown, a proximal hub may be secured to the proximal region 38 of the elongate shaft 32.

In some cases, as noted, the expandable element 34 may be an inflatable balloon, and thus may include a distal waist 42 and a proximal waist 44. In some cases, the distal waist 42 may be secured relative to a distal end of the guidewire lumen 40 while the proximal waist 44 may be secured relative to a distal end of the elongate shaft 32. In some cases, an atraumatic tip 46 may be disposed where the distal waist 42 is secured relative to the distal end of the guidewire lumen 40. An inflation lumen (not shown) extending through the elongate shaft 32 may be fluidly coupled with an interior volume of the expandable element 34 such that an inflation fluid such as saline may be pumped or otherwise provided to the interior volume in order to cause the expandable element 34 to move from its collapsed configuration (as shown in FIG. 2A) to its expanded configuration (as shown in FIG. 2B). After being expanded, or inflated, the expandable element 34 may subsequently be deflated by withdrawing the inflation fluid from within the interior volume of the expandable element 34.

The medical device 30 includes a conductive member 48. In some cases, the conductive member 48 may include an electrocautery electrode or electrocautery electrode portion 50 that corresponds to a portion of the conductive member 48 that extends over the expandable element 34. It will be appreciated that a length of the electrocautery electrode or electrocautery electrode portion 50 may vary, depending on whether the expandable element 34 is deflated or inflated. As an example, the electrocautery electrode or electrocautery electrode portion 50 shown in FIG. 2A, with the expandable element 34 deflated, may be considered as having a first length. The electrocautery electrode or electrocautery electrode portion 50 shown in FIG. 2B, with the expandable element 34 inflated, may be considered as having a second length that is greater than the first length. This assumes, of course, that the electrocautery electrode or electrocautery electrode portion 50 is contoured to an outer surface 54 of the expandable element 34. The additional length (i.e., the difference between the second length and the first length) needs to be accounted for in order to keep the elongate shaft 32 in a desired configuration. In some cases, the conductive member 48 may be adapted to accommodate this change in length and thus provide axial stability to the medical device 30. Absent an ability to accommodate a change in length, the elongate shaft 32 may instead be caused to bend when the expandable element 34 is inflated or otherwise expanded.

In some cases, the conductive member 48 may include a biasing member 52. In some cases, the biasing member 52 may be an integrally formed part of the conductive member 48. In some cases, the biasing member 52 may be separately formed and then secured to the rest of the conductive member 48. Similarly, in some cases, the electrocautery electrode or electrocautery electrode portion 50 may be an integrally formed part of the conductive member 48. In some cases, the electrocautery electrode or electrocautery electrode portion 50 may be separately formed and then secured to the rest of the conductive member 48.

As shown in FIGS. 2A and 2B, the biasing member 52 may be an undulating region 56 that is part of the conductive member 48. The undulating region 56 may be considered as having a “remembered” configuration, as shown for example in FIG. 2A. The undulating region 56 may be biased into the configuration shown in FIG. 2A. Exerting a tensile force on the conductive member 48, such as by inflating the expandable element 34, will cause the undulating region 56 to lengthen, as shown for example in FIG. 2B. The undulating region 56 lengthening provides the difference between the first length and the second length, for example. In some cases, when the tensile force on the conductive member 48 is removed, such as by deflating the expandable element 34, the undulating region 56 may revert to its original configuration, thereby eliminating any excess length of the conductive member 48.

In some cases, the conductive member 48 may extend within a lumen formed within the elongate shaft 32. In some cases, the conductive member 48 may extend along an exterior of the elongate shaft 32. While FIG. 2A shows the undulating region 56 as having a constant or largely constant outer diameter (defined by the extent the conductive member 48 extends in any direction from an imaginary central axis), this is not required in all cases. In some cases, the undulating region 56 may instead have a tapering diameter in which the outer diameter increases proximally to distally. In some cases, the undulating region 56 may have a tapering diameter in which the outer diameter decreases proximally to distally. In some cases, the electrocautery electrode or electrocautery electrode portion 50 may have a distal end that is secured at a securement point 58. The securement point 58 may be part of the atraumatic tip 46, for example.

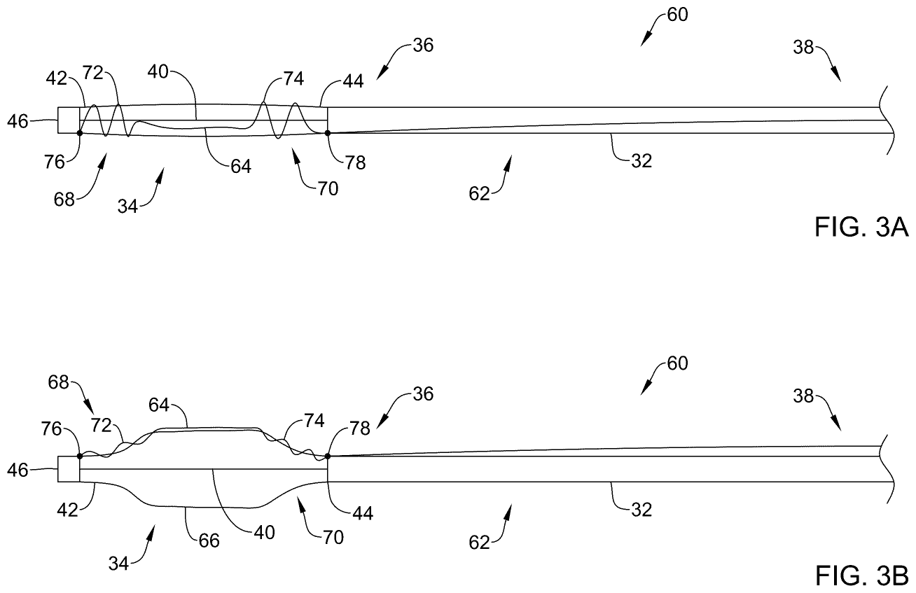

FIGS. 2A and 2B show a biasing member 52 that includes a single undulating region 56. In some cases, the biasing member 52 may include more than one undulating region 56. FIGS. 3A and 3B are schematic views of a medical device 60. The medical device 60 may be considered as being usable in place of the medical device 30. FIG. 3A shows the expandable element 34 in a collapsed configuration and FIG. 3B shows the expandable element 34 in an expanded configuration. As before, the elongate shaft 32 includes the distal region 36 and extends proximally to the proximal region 38. The expandable element 34 may be secured to the distal region 36 of the elongate shaft 32. In some cases, a guidewire lumen 40 may be seen as extending distally of the elongate shaft 32 and extending through the expandable element 34. While not expressly shown, it will be appreciated that the guidewire lumen 40 may extend proximally through the elongate shaft 32. In some cases, additional lumens (not shown) may also extend through the elongate shaft 32. As an example, an inflation lumen may extend through the elongate shaft 32, particularly when the expandable element 34 is an inflatable balloon. In some cases, while not shown, a proximal hub may be secured to the proximal region 38 of the elongate shaft 32.

In some cases, as noted, the expandable element 34 may be an inflatable balloon, and thus may include the distal waist 42 and the proximal waist 44. In some cases, the distal waist 42 may be secured relative to a distal end of the guidewire lumen 40 while the proximal waist 44 may be secured relative to a distal end of the elongate shaft 32. In some cases, the atraumatic tip 46 may be disposed where the distal waist 42 is secured relative to the distal end of the guidewire lumen 40. An inflation lumen (not shown) extending through the elongate shaft 32 may be fluidly coupled with an interior volume of the expandable element 34 such that an inflation fluid such as saline may be pumped or otherwise provided to the interior volume in order to cause the expandable element 34 to move from its collapsed configuration (as shown in FIG. 3A) to its expanded configuration (as shown in FIG. 3B). After being expanded, or inflated, the expandable element 34 may subsequently be deflated by withdrawing the inflation fluid from within the interior volume of the expandable element 34.

The medical device 60 includes a conductive member 62. In some cases, the conductive member 62 may include an electrocautery electrode or electrocautery electrode portion 64 that corresponds to a portion of the conductive member 62 that extends over the expandable element 34. It will be appreciated that a length of the electrocautery electrode or electrocautery electrode portion 64 may vary, depending on whether the expandable element 34 is deflated or inflated. As an example, the electrocautery electrode or electrocautery electrode portion 64 shown in FIG. 3A, with the expandable element 34 deflated, may be considered as having a first length. The electrocautery electrode or electrocautery electrode portion 64 shown in FIG. 3B, with the expandable element 34 inflated, may be considered as having a second length that is greater than the first length. This assumes, of course, that the electrocautery electrode or electrocautery electrode portion 64 is contoured to an outer surface 66 of the expandable element 34. The additional length (i.e., the difference between the second length and the first length) needs to be accounted for in order to keep the elongate shaft 32 in a desired configuration. In some cases, the conductive member 62 may be adapted to accommodate this change in length.

In some cases, the conductive member 62 may include a first biasing member 68 and a second biasing member 70. In some cases, the first biasing member 68 and the second biasing member 70 may each be integrally formed parts of the conductive member 62. In some cases, the first biasing member 68 and the second biasing member 70 may each be separately formed and then secured to the rest of the conductive member 62. Similarly, in some cases, the electrocautery electrode or electrocautery electrode portion 64 may be an integrally formed part of the conductive member 62. In some cases, the electrocautery electrode or electrocautery electrode portion 64 may be separately formed and then secured to the rest of the conductive member 62.

As shown in FIGS. 3A and 3B, the first biasing member 68 may be a first undulating region 72 that is part of the conductive member 62 and the second biasing member 70 may be a second undulating region 74 that is part of the conductive member 62. Each of the first undulating region 72 and the second undulating region 74 may be considered as having a “remembered” configuration, as shown for example in FIG. 3A. The first undulating region 72 and the second undulating region 74 may each be biased into the configuration shown in FIG. 3A. Exerting a tensile force on the conductive member 62, such as by inflating the expandable element 34, will cause the first undulating region 72 and the second undulating region 74 to lengthen, as shown for example in FIG. 3B. The first undulating region 72 and the second undulating region 74 each lengthening provides the difference between the first length and the second length, for example. In some cases, when the tensile force on the conductive member 62 is removed, such as by deflating the expandable element 34, the first undulating region 72 and the second undulating region 74 may each revert to their original configuration, thereby eliminating any excess length of the conductive member 62.

In some cases, the conductive member 62 may extend within a lumen formed within the elongate shaft 32. In some cases, the conductive member 62 may extend along an exterior of the elongate shaft 32. While FIG. 3A shows the first undulating region 72 and the second undulating region 74 as having a constant or largely constant outer diameter (defined by the extent the conductive member 62 extends in any direction from an imaginary central axis), this is not required in all cases. In some cases, the first undulating region 72 and/or the second undulating region 74 may independently have a tapering diameter in which the outer diameter increases proximally to distally. In some cases, the first undulating region 72 and the second undulating region 74 may independently have a tapering diameter in which the outer diameter decreases proximally to distally.

In some cases, the first undulating region 72 and/or the second undulating region 74 may be electrically active, and thus may be considered as being part of the electrocautery electrode or electrocautery electrode portion 64. In some cases, the first undulating region 72 and/or the second undulating region 74 may not electrically active, or may be electrically insulated, and thus may not be considered as part of the electrocautery electrode or electrocautery electrode portion 64. In some cases, the first undulating region 72 may include a distal end that is secured at a first securement point 76. The first securement point 76 may be part of the atraumatic tip 46, for example. The second undulating region 74 may have a proximal end that is secured at a second securement point 76. The second securement point 76 may be at or near a distal end of the elongate shaft 32.

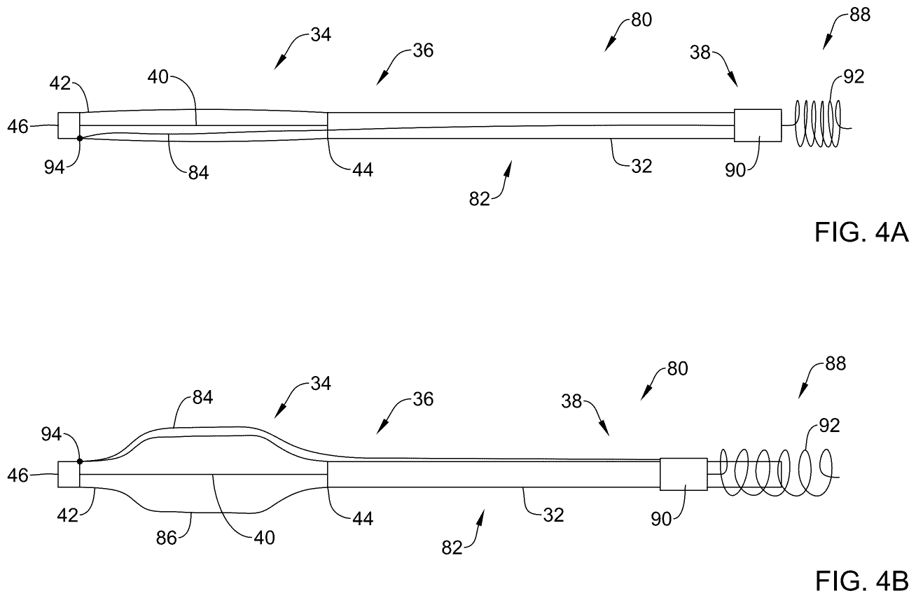

FIG. 4A and FIG. 4B are schematic views of a medical device 80. The medical device 80 may be considered as being usable in place of the medical device 30. FIG. 4A shows the expandable element 34 in a collapsed configuration and FIG. 4B shows the expandable element 34 in an expanded configuration. The elongate shaft 32 includes the distal region 36 and extends proximally to the proximal region 38. The expandable element 34 may be secured to the distal region 36 of the elongate shaft 32. In some cases, the guidewire lumen 40 may be seen as extending distally of the elongate shaft 32 and extending through the expandable element 34. While not expressly shown, it will be appreciated that the guidewire lumen 40 may extend proximally through the elongate shaft 32. In some cases, additional lumens (not shown) may also extend through the elongate shaft 32. As an example, an inflation lumen may extend through the elongate shaft 32, particularly when the expandable element 34 is an inflatable balloon. In some cases, while not shown, a proximal hub may be secured to the proximal region 38 of the elongate shaft 32.

In some cases, as noted, the expandable element 34 may be an inflatable balloon, and thus may include the distal waist 42 and the proximal waist 44. In some cases, the distal waist 42 may be secured relative to a distal end of the guidewire lumen 40 while the proximal waist 44 may be secured relative to a distal end of the elongate shaft 32. In some cases, the atraumatic tip 46 may be disposed where the distal waist 42 is secured relative to the distal end of the guidewire lumen 40. An inflation lumen (not shown) extending through the elongate shaft 32 may be fluidly coupled with an interior volume of the expandable element 34 such that an inflation fluid such as saline may be pumped or otherwise provided to the interior volume in order to cause the expandable element 34 to move from its collapsed configuration (as shown in FIG. 4A) to its expanded configuration (as shown in FIG. 4B). After being expanded, or inflated, the expandable element 34 may subsequently be deflated by withdrawing the inflation fluid from within the interior volume of the expandable element 34.

The medical device 80 includes a conductive member 82. In some cases, the conductive member 82 may include an electrocautery electrode or electrocautery electrode portion 84 that corresponds to a portion of the conductive member 82 that extends over the expandable element 34. It will be appreciated that a length of the electrocautery electrode or electrocautery electrode portion 84 may vary, depending on whether the expandable element 34 is deflated or inflated. As an example, the electrocautery electrode or electrocautery electrode portion 84 shown in FIG. 4A, with the expandable element 34 deflated, may be considered as having a first length. The electrocautery electrode or electrocautery electrode portion 84 shown in FIG. 4B, with the expandable element 34 inflated, may be considered as having a second length that is greater than the first length. This assumes, of course, that the electrocautery electrode or electrocautery electrode portion 84 is contoured to an outer surface 86 of the expandable element 34. The additional length (i.e., the difference between the second length and the first length) needs to be accounted for in order to keep the elongate shaft 32 in a desired configuration. In some cases, the conductive member 82 may be adapted to accommodate this change in length.

In some cases, the medical device 80 may include a biasing member 88, and the conductive member 82 may be operably coupled with the biasing member 88 such that the biasing member 88 is able to change a length of the conductive member 82 and thus accommodate changes in length of the electrocautery electrode or electrocautery electrode portion 84 when the expandable element 34 is inflated or deflated. In some cases, the biasing member 88 may include a sled 90 that is slidingly disposed relative to the elongate shaft 32. The conductive member 82 may be operably coupled to the sled 90. The biasing member 88 may include a spring 92 that biases the sled 90 to a proximal position, as shown in FIG. 4A. When a tensile force is applied via the conductive member 82 to the sled 90, the sled 90 is able to move distally against a biasing force provided by the spring 92, as shown in FIG. 4B. The spring 92 may elongate from its resting position shown in FIG. 4A. This accommodates the additional length in the electrocautery electrode or electrocautery electrode portion 84 when the expandable element 34 is inflated or deflated.

In some cases, the conductive member 82, including the biasing member 88, may extend within a lumen formed within the elongate shaft 32. In some cases, the conductive member 82, including the biasing member 88, may extend along an exterior of the elongate shaft 32. In some cases, the electrocautery electrode or electrocautery electrode portion 84 may have a distal end that is secured at a securement point 94. The securement point 94 may be part of the atraumatic tip 46, for example.

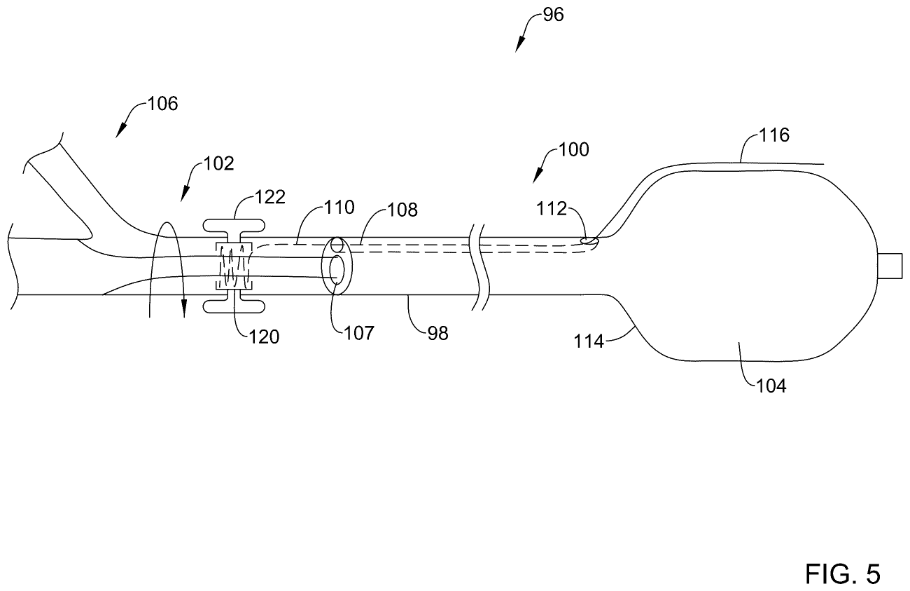

FIG. 5 is a schematic view of a medical device 96. The medical device 96 may be considered as being usable in place of the medical device 30. The medical device 96 includes an elongate shaft 98 that extends between a distal region 100 and a proximal region 102. An inflatable balloon 104 is secured to the distal region 100 of the elongate shaft 98. A proximal hub 106 is secured to the proximal region 102 of the elongate shaft 98. In some cases, the elongate shaft 98 may include multiple lumens. As an example, the elongate shaft 98 may include a central shaft 107 that may be used for tracking the medical device 96 over a guidewire (not shown). The elongate shaft 98 may include a conductive member lumen 108 for accommodating a conductive member 110. The conductive member 110 may exit the elongate shaft 98 through an aperture 112 that is proximal of a proximal balloon waist 114 of the inflatable balloon 104 and may include or be connected to an electrocautery electrode or electrocautery electrode portion 116. The elongate shaft 98 may also include an annular inflation shaft (not shown) that allows an inflation fluid such as saline to be provided to the inflatable balloon 104.

The proximal hub 106 may include a spool 120 that is disposed within the proximal hub 106 and is rotatable relative to the proximal hub 106. In some cases, the spool 120 may be considered as being a biasing member. A portion of the conductive member 110 is spooled on the spool 120. As the inflatable balloon 104 is inflated, a length of the electrocautery electrode or electrocautery electrode portion 116 will lengthen to accommodate the increased diameter of the inflatable balloon 104. As the electrocautery electrode or electrocautery electrode portion 116 lengthens, the spool 120 will rotate relative to the proximal hub 106, thereby allowing additional conductive member 110 to unspool. In some cases, the proximal hub 106 may include a mounting feature 122 that serves to locate the spool 120 relative to the proximal hub 106.

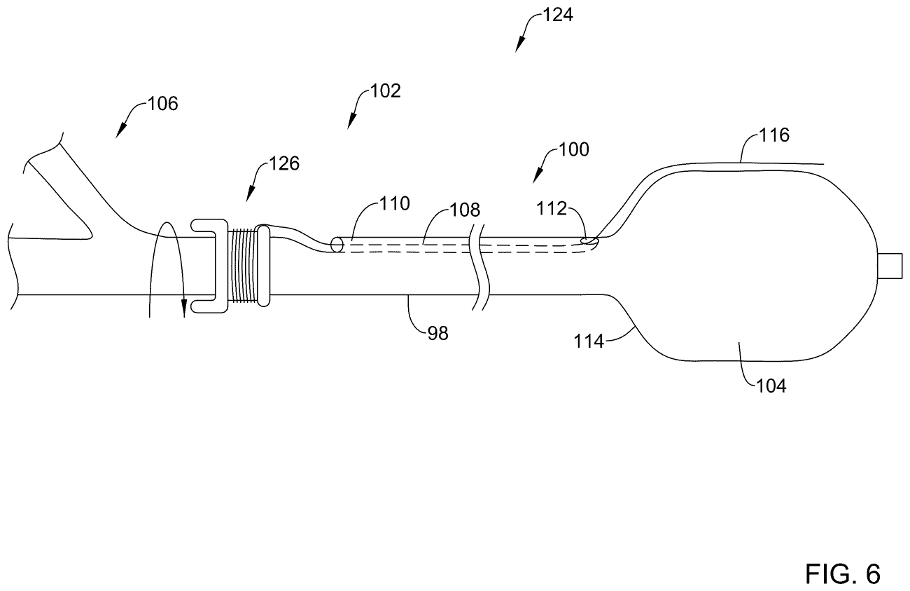

FIG. 6 is a schematic view of a medical device 124. The medical device 124 may be considered as being usable in place of the medical device 30. The medical device 124 includes an elongate shaft 98 that extends between the distal region 100 and the proximal region 102. The inflatable balloon 104 is secured to the distal region 100 of the elongate shaft 98. The proximal hub 106 is secured to the proximal region 102 of the elongate shaft 98. As noted with respect to FIG. 5, the elongate shaft 98 may include multiple lumens. The elongate shaft 98 may include the conductive member lumen 108 for accommodating the conductive member 110. The conductive member 110 may exit the elongate shaft 98 through the aperture 112 that is proximal of the proximal balloon waist 114 of the inflatable balloon 104 and may include or be connected to an electrocautery electrode or electrocautery electrode portion 116.

The proximal hub 106 may include a spool 126 that is disposed about the proximal hub 106 and is rotatable relative to the proximal hub 106. In some cases, the spool 126 may be considered as being a biasing member. A portion of the conductive member 110 is spooled on the spool 126. As the inflatable balloon 104 is inflated, a length of the electrocautery electrode or electrocautery electrode portion 116 will lengthen to accommodate the increased diameter of the inflatable balloon 104. As the electrocautery electrode or electrocautery electrode portion 116 lengthens, the spool 126 will rotate relative to the proximal hub 106, thereby allowing additional conductive member 110 to unspool. In some cases, the spool 126 may be manually activated or spring-activated. The spool 126 may be magnetically or electromagnetically activated or biased to recoil the conductive member 110. In some cases, the spool 126 may act to recoil the conductive member 110 in response to either a rotary or axial movement.

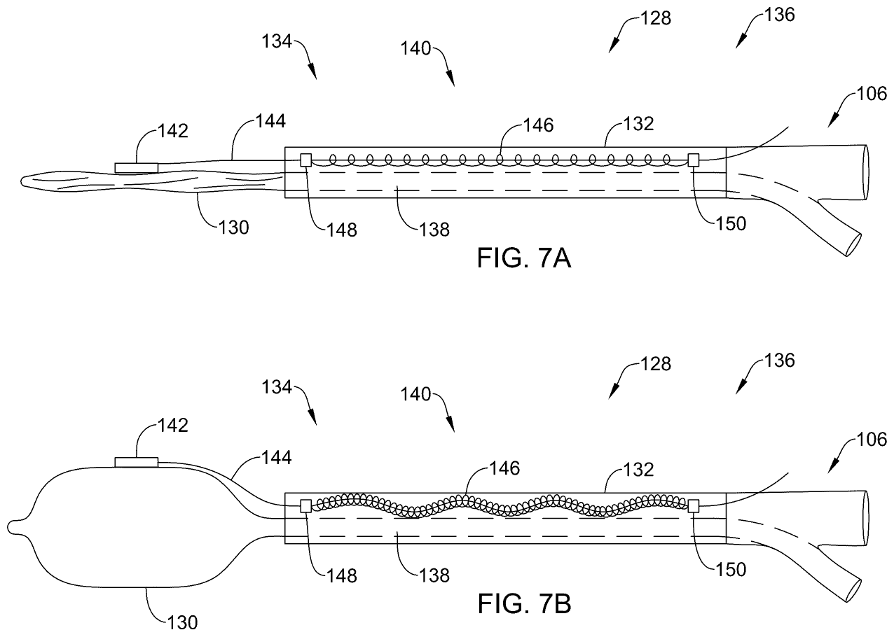

FIG. 7A and FIG. 7B are schematic views of an illustrative medical device 128. The medical device 128 may be considered as being usable in place of the medical device 30. FIG. 7A shows the medical device 128 with an inflatable balloon 130 in a deflated configuration and FIG. 7B shows the medical device 128 with the inflatable balloon 130 in an inflated configuration. The medical device 128 includes an elongate shaft 132 that extends from a distal region 134 to a proximal region 136. The inflatable balloon 130 is secured to the distal region 134 of the elongate shaft 132. The proximal hub 106 is secured to the proximal region 136 of the elongate shaft 132. An inflation lumen 138 extends through the elongate shaft 132 from the proximal hub 106 to an interior of the inflatable balloon 130.

A conductive member 140 includes an electrocautery electrode or electrocautery electrode portion 142 that is disposed on an exterior of the inflatable balloon 130. The conductive member 140 also includes a wire 144 that extends proximally from the electrocautery electrode or electrocautery electrode portion 142. Part of the wire 144 extends within a coil 146 that is disposed relative to the elongate shaft 132. The coil 146 is secured relative to the elongate shaft 132 via a distal stop 148 and a proximal stop 150. The coil 146 is biased to an open coil configuration as shown in FIG. 7A. When the inflatable balloon 130 inflates, the coil 146 recoils tightly as the wire 144 is pulled through the coil 146 in order to accommodate a length change for the electrocautery electrode or electrocautery electrode portion 142.

The materials that can be used for the various components of the medical devices and the various elements thereof disclosed herein may include those commonly associated with medical devices. In some instances, the medical assembly 10, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamidc/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; silver; combinations thereof; or any other suitable material.

In at least some instances, portions or all of the medical assembly 10, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical devices, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some instances, the medical devices disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device for lacerating valve leaflets, the medical device comprising:
an elongate shaft extending proximally from a distal region;
an expandable element secured within the distal region, the expandable element is movable between a collapsed configuration and an expanded configuration; and
a conductive member including an electrocautery electrode portion extending over the expandable element, the electrocautery electrode portion having a first length when the expandable element is in the collapsed configuration and a second length greater than the first length when the expandable element is in its expanded configuration;
wherein the conductive member is adapted to return the electrocautery electrode portion to the first length when the expandable element returns to the collapsed configuration.

2. The medical device of claim 1, wherein the conductive member comprises an undulating region that is adapted to elongate in order to accommodate a change from the first length of the electrocautery electrode portion to the second length of the electrocautery electrode portion when the expandable element expands into the expanded configuration.

3. The medical device of claim 2, wherein the undulating region is adapted to contract in order to accommodate a change from the second length of the electrocautery electrode portion to the first length of the electrocautery electrode when the expandable element returns to the collapsed configuration.

4. The medical device of claim 1, wherein a distal region of the electrocautery electrode portion is secured to a distal region of the expandable element.

5. The medical device of claim 4, wherein the undulating region is disposed proximal of the expandable element.

6. The medical device of claim 1, wherein a distal region of the electrocautery electrode portion is secured to a distal region of the expandable element and a proximal region of the electrocautery electrode portion is secured to a proximal region of the expandable element.

7. The medical device of claim 6, wherein the conductive member comprises:
a first undulating region adjacent to the distal region of the electrocautery electrode portion; and
a second undulating region adjacent to the proximal region of the electrocautery electrode portion.

8. The medical device of claim 1, further comprising:
a sled slidingly disposed relative to the elongate shaft;
a spring biasing the sled to a proximal position;
wherein the conductive member is operably coupled with the sled such that the sled moves distally against a biasing force provided by the spring when the expandable element expands into the expanded configuration.

9. The medical device of claim 8, wherein the sled moves proximally when the expandable element returns to the collapsed configuration.

10. The medical device of claim 1, further comprising:
a proximal hub secured to a proximal region of the elongate shaft; and
a spool disposed relative to the proximal hub, the spool is adapted to releasably wind up a portion of the conductive member on the spool.

11. The medical device of claim 10, wherein the spool is disposed inside the proximal hub.

12. The medical device of claim 10, wherein the spool is disposed outside of the proximal hub.

13. The medical device of claim 1, wherein the expandable element comprises an inflatable balloon.

14. A medical device for lacerating valve leaflets, the medical device comprising:
an elongate shaft extending proximally from a distal region;
an inflatable balloon secured within the distal region and adapted to be inflated and/or deflated;
an electrocautery electrode that extends over the inflatable balloon;
a conductive member extending proximally from the electrocautery electrode; and
a biasing member that is adapted to accommodate a change in length of the electrocautery electrode when the inflatable balloon is either inflated or deflated.

15. The medical device of claim 14, wherein the biasing member comprises an undulating region of the conductive member.

16. The medical device of claim 15, wherein the biasing member further includes a second undulating region of the conductive member.

17. The medical device of claim 14, wherein the biasing member comprises:
a sled slidingly disposed relative to the elongate shaft; and
a spring biasing the sled to a proximal position;
wherein the conductive member is operably coupled with the sled such that the sled moves distally against a biasing force provided by the spring when the inflatable balloon is inflated.

18. The medical device of claim 14, further comprising:
a proximal hub secured to a proximal region of the elongate shaft;
wherein the biasing member comprises a spool disposed relative to the proximal hub and adapted to releasably wind up a portion of the conductive member on the spool.

19. A medical device for lacerating valve leaflets, the medical device comprising:
an elongate shaft extending proximally from a distal region;
an inflatable balloon secured within the distal region and adapted to be inflated and/or deflated; and
a conductive member extending relative to the elongate shaft, the conductive member including:
an electrocautery electrode portion that extends over the inflatable balloon; and
an undulating region that is adapted to elongate in order to accommodate an increase in length of the electrocautery electrode portion when the inflatable balloon is inflated.

20. The medical device of claim 19, wherein the undulating region is adapted to decrease in length in order to accommodate a decrease in length of the electrocautery electrode portion when the inflatable balloon is deflated.
